(19) United States
(12) Patent Application Publication
(10) Pub. No.: US 2016/0143237 A1
(43) Pub. Date: May 26, 2016

(54) METHODS FOR IMPROVING PLANT TISSUE CULTURE

(71) Applicant: E I DU PONT DE NEMOURS AND COMPANY, Wilmington, DE (US)

(72) Inventors: Jyothi P. Bolar, Hockessin, DE (US); Cheryl P. Caster, Landenberg, PA (US); Christian F. Lochetto, Newtown, PA (US); Richard P. Mccardell, Landenberg, PA (US); Alessandro Pellegrineschi, Wilmington, DE (US)

(73) Assignee: E I DU PONT DE NEMOURS AND COMPANY, Wilmington, DE (US)

(21) Appl. No.: 14/132,441

(22) Filed: Dec. 18, 2013

Related U.S. Application Data
(60) Provisional application No. 61/748,028, filed on Dec. 31, 2012.

(51) Int. Cl. A01H 4/00

(52) U.S. Cl. CPC A01H 4/001

(57) ABSTRACT

The invention relates to the improvement of plant tissue culture. The invention relates to a method for improving the efficiency of response of plant tissues to tissue culture. It specifically relates to using vacuum infiltration to introduce media and other solutions into the plant tissues.

### CROSS REFERENCE TO RELATED APPLICATIONS

This application claims the benefit of U.S. Provisional Application No. 61/748,028, filed Dec. 31, 2012, the entire content of which is herein incorporated by reference.

### FIELD OF INVENTION

The present invention relates to the improvement of plant tissue culture. It specifically relates to using vacuum infiltration to introduce media and other solutions into the plant tissues.

### BACKGROUND

Recent advances in plant genetic engineering have opened new doors to engineer plants to have improved characteristics or traits. Crop improvement via genetic transformation requires suitable recipient tissue for gene transfer followed by regeneration of transformed plants. Almost all plant transformation methods rely on tissue culture at some step. The ability to regenerate plants from isolated cells or tissues in vitro underpins most plant transformation systems. Most methods of plant transformation used for genetically modifying crops require that a whole plant is regenerated from isolated plant cells or tissues that have been genetically transformed. This regeneration is conducted in vitro so that the environment and growth medium can be manipulated to ensure a high frequency of regeneration. (U.S. Pat. No. 6,492,174; Grewal, D. et al. 2006 Biotechnol J. 1(10):1158-62).

Soybean is a major crop worldwide, which contributes significantly to the global economy as an important source of dietary oil and protein (Wilcox, 2004). Therefore, improvement of soybean characteristics via regeneration and transformation techniques is of critical importance. Although soybean is one of the most recalcitrant crops (Hofmann et al. (2004), Plant Cell, Tissue and Organ Culture 77: 157-163; Trick et al. (1997) Plant Tissue Culture and Biotechnology 3 (1):9-26); Paz et al. (2006) Plant Cell Rep 25: 206-213), and many soybean regeneration and transformation methods have been established over the years, the efficiency of these techniques is considerably restricted by low efficiency, poor reproducibility and genotype dependence (Paz et al., 2004; Reichert et al., 2003; Sairam et al., 2003; Yang et al., 2009). One of the critical prerequisites for all current soybean transformation procedures is the ability to manipulate plant tissue in vitro.

### SUMMARY

An embodiment of this invention is a method for increasing efficiency of response of plant tissue to tissue culture, the method comprising the steps of: (a) contacting a plant tissue with liquid media wherein the liquid media comprises at least one constituent selected from the group consisting of nutrients, growth regulators and antibiotics; (b) subjecting the plant tissue and liquid media of the previous step to a partial vacuum, followed by (c) returning the plant tissue and liquid media of step to atmospheric pressure; wherein there is an increased efficiency of response when compared to a plant tissue that is not subjected to a partial vacuum and further wherein the response of plant tissue can be selected from a group consisting of embryogenesis, organogenesis and cell division. In one embodiment of the invention, the liquid media contains an antibiotic and the plant tissue is not contaminated with bacteria. In one embodiment of this invention, the efficiency of response of plant tissue to tissue culture is increased by at least 30% when compared to the response of a plant tissue in tissue culture that has not been subject to partial vacuum after being contacted with liquid media.

Another embodiment of this invention is a method for increasing synchronization of development of plant tissue in tissue culture, the method comprising steps (a), (b) and (c) described above, wherein there is an increase in synchronization of development when compared to a plant tissue that is not subjected to a partial vacuum.

Another embodiment of this invention is a method for increasing efficiency of regeneration of a mature and fertile plant from plant tissue in tissue culture, the method comprising steps (a), (b) and (c) described above, wherein there is an increase in efficiency of regeneration when compared to a plant tissue that is not subjected to a partial vacuum.

Another embodiment of this invention is the method of increasing efficiency of response of plant tissue to tissue culture, or the method of increasing synchronization of development of plant tissue in tissue culture, or the method of increasing efficiency of regeneration of a mature and fertile plant from plant tissue in tissue culture, the method comprising the steps described above, wherein the method further comprises the step of introducing a recombinant DNA construct into the plant tissue. Furthermore, the recombinant DNA may be introduced into the plant tissue by contacting the plant tissue with Agrobacterium, or by particle bombardment.

Another embodiment of this invention is the method of increasing efficiency of response of plant tissue to tissue culture, or the method of increasing synchronization of development of plant tissue in tissue culture, or the method of increasing efficiency of regeneration of a mature and fertile plant from plant tissue in tissue culture, wherein the plant tissue is recalcitrant to tissue culture.

In one embodiment, the methods disclosed in the current invention are used for increasing the tissue culture response of soybean plant tissues.

In another embodiment, the invention includes plant cells, tissues, plants, and seeds generated by using the methods disclosed in the current invention. The invention encompasses regenerated, mature and fertile plants, seeds produced therefrom, T1 and subsequent generations. The plant cells, tissues, plants and seeds may be soybean cells, tissues, plants and seeds.

### BRIEF DESCRIPTION OF DRAWINGS

The invention can be more fully understood from the following detailed description and the accompanying drawings which form a part of this application.

FIG. 1A-FIG. 1B show the effects of media vacuum infiltration on somatic embryogenesis.

FIG. 1A shows the somatic embryogenesis after media vacuum infiltration and FIG. 1B shows the somatic embryogenesis in the control samples.

FIG. 2A-FIG. 2D show quantitative analysis of the effects of media infiltration on somatic embryogenesis.

FIG. 2A shows the boxplot for percentage of embryogenic tissue produced in the presence or absence of vacuum infiltration.

FIG. 2B-FIG. 2D shows the summary for percentage of embryogenic tissue in presence or absence of vacuum infiltration.

FIG. 2B shows a histogram of data with an overlaid normal curve.

FIG. 2C shows the 95% confidence intervals for the mean, and 95% confidence intervals for the median.

FIG. 2D shows a table of Anderson-Darling statistic that measures how well the data follow a particular distribution. The better the distribution fits the data, the smaller this statistic will be. Below this there are some descriptive statistics and on the last box (Confidence interval) is represented a range of values, derived from sample statistics that is likely to contain the value of an unknown population parameter. Because of their random nature, it is unlikely that two samples from a given population will yield identical confidence intervals.

FIG. 3A and FIG. 3B show the interaction and cube plots to identify the best combination between Carbeniciilin, Cefotaxime and time.

FIG. 3A shows the interaction plot to identify the best combination between Carbenicillin, Cefotaxime and time to obtain the maximum percentage response of least contamination.

FIG. 3B shows the cube plot to identify the best combination between Carbeniciilin, Cefotaxime and time to obtain the maximum percentage response of least contamination.

FIG. 4A-FIG. 4E show the bacterial contamination response after vacuum infiltration with the antibiotics.

FIG. 4A and FIG. 4B show the results with standard plating and with vacuum infiltration only, respectively.

FIG. 4C shows the results when the samples are vacuum infiltrated with media containing 50 mg/I carbeniciilin and 10 mg/l Cefotaxime.

FIG. 4D shows the results when the samples are vacuum infiltrated with media containing 75 mg/I carbeniciilin and 30 mg/l Cefotaxime.

FIG. 4E shows the results when the samples are vacuum infiltrated with media containing 100 mg/I carbeniciilin and 50 mg/l Cefotaxime.

### DETAILED DESCRIPTION OF THE INVENTION

The disclosure of each reference set forth herein is hereby incorporated by reference in its entirety.

As used herein and in the appended claims, the singular forms “a”, “an”, and “the” include plural reference unless the context clearly dictates otherwise. Thus, for example, reference to “a plant” includes a plurality of such plants, reference to “a cell” includes one or more cells and equivalents thereof known to those skilled in the art, and so forth.

As used herein:

The terms “monocot” and “monocotyledonous plant” are used interchangeably herein. A monocot of the current invention includes the Gramineae.

The terms “dicot” and “dicotyledonous plant” are used interchangeably herein. A dicot of the current invention includes the following families: Brassicaceae, Leguminosae, and Solanaceae.

The terms “full complement” and “full-length complement” are used interchangeably herein, and refer to a complement of a given nucleotide sequence, wherein the complement and the nucleotide sequence consist of the same number of nucleotides and are 100% complementary.

“Transgenic” refers to any cell, cell line, callus, tissue, plant part or plant, the genome of which has been altered by the presence of a heterologous nucleic acid, such as a recombinant DNA construct, including those initial transgenic events as well as those created by sexual crosses or asexual propagation from the initial transgenic event. The term “transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition, or spontaneous mutation. “Genome” as it applies to plant cells encompasses not only chromosomal DNA found within the nucleus, but organelle DNA found within subcellular components (e.g., mitochondrial, plastid) of the cell.

“Plant” includes reference to whole plants, plant organs, plant tissues, seeds and plant cells and progeny of same. Plant cells include, without limitation, cells from seeds, suspension cultures, embryos, meristematic regions, callus tissue, leaves, roots, shoots, gametophytes, sporophytes, pollen, and microspores.

“Progeny” comprises any subsequent generation of a plant.

“Transgenic plant” includes reference to a plant which comprises within its genome a heterologous polynucleotide. For example, the heterologous polynucleotide is stably integrated within the genome such that the polynucleotide is passed on to successive generations. The heterologous polynucleotide may be integrated into the genome alone or as part of a recombinant DNA construct.

“Heterologous” with respect to sequence means a sequence that originates from a foreign species, or, if from the same species, is substantially modified from its native form in composition and/or genomic locus by deliberate human intervention.

“Polynucleotide”, “nucleic acid sequence”, “nucleotide sequence”, or “nucleic acid fragment” are used interchangeably to refer to a polymer of RNA or DNA that is single- or double-stranded, optionally containing synthetic, non-natural or altered nucleotide bases. Nucleotides (usually found in their 5′-monophosphate form) are referred to by their single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide.

“Polypeptide”, “peptide”, “amino acid sequence” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The terms “polypeptide”, “peptide”, “amino acid sequence”, and “protein” are also inclusive of modifications including, but not limited to, glycosylation, lipid attachment, sulfation, gamma-carboxylation of glutamic acid residues, hydroxylation and ADP-ribosylation.

“Messenger RNA (mRNA)” refers to the RNA that is without introns and that can be translated into protein by the cell.

“cDNA” refers to a DNA that is complementary to and synthesized from an mRNA template using the enzyme reverse transcriptase. The cDNA can be single-stranded or converted into the double-stranded form using the Klenow fragment of DNA polymerase I.

An “Expressed Sequence Tag” (“EST”) is a DNA sequence derived from a cDNA library and therefore is a sequence which has been transcribed. An EST is typically obtained by a single sequencing pass of a cDNA insert. The sequence of an entire cDNA insert is termed the “Full-Insert Sequence” (“FIS”). A “Contig” sequence is a sequence assembled from two or more sequences that can be selected from, but not limited to, the group consisting of an EST, FIS and PCR sequence. A sequence encoding an entire or functional protein is termed a “Complete Gene Sequence” (“CGS”) and can be derived from an FIS or a contig.

“Mature” protein refers to a post-translationally processed polypeptide; i.e., one from which any pre- or pro-peptides present in the primary translation product has been removed.

“Precursor” protein refers to the primary product of translation of mRNA; i.e., with pre- and pro-peptides still present. Pre- and pro-peptides may be and are not limited to intracellular localization signals.

“Isolated” refers to materials, such as nucleic acid molecules and/or proteins, which are substantially free or otherwise removed from components that normally accompany or interact with the materials in a naturally occurring environment. Isolated polynucleotides may be purified from a host cell in which they naturally occur. Conventional nucleic acid purification methods known to skilled artisans may be used to obtain isolated polynucleotides. The term also embraces recombinant polynucleotides and chemically synthesized polynucleotides.

“Recombinant” refers to an artificial combination of two otherwise separated segments of sequence, e.g., by chemical synthesis or by the manipulation of isolated segments of nucleic acids by genetic engineering techniques. “Recombinant” also includes reference to a cell or vector, that has been modified by the introduction of a heterologous nucleic acid or a cell derived from a cell so modified, but does not encompass the alteration of the cell or vector by naturally occurring events (e.g., spontaneous mutation, natural transformation/transduction/transposition) such as those occurring without deliberate human intervention.

“Recombinant DNA construct” refers to a combination of nucleic acid fragments that are not normally found together in nature. Accordingly, a recombinant DNA construct may comprise regulatory sequences and coding sequences that are derived from different sources, or regulatory sequences and coding sequences derived from the same source, but arranged in a manner different than that normally found in nature.

The terms “entry clone” and “entry vector” are used interchangeably herein.

“Operably linked” refers to the association of nucleic acid fragments in a single fragment so that the function of one is regulated by the other. For example, a promoter is operably linked with a nucleic acid fragment when it is capable of regulating the transcription of that nucleic acid fragment.

“Expression” refers to the production of a functional product. For example, expression of a nucleic acid fragment may refer to transcription of the nucleic acid fragment (e.g., transcription resulting in mRNA or functional RNA) and/or translation of mRNA into a precursor or mature protein.

“Overexpression” refers to the production of a gene product in transgenic organisms that exceeds levels of production in a null segregating (or non-transgenic) organism from the same experiment.

“Phenotype” means the detectable characteristics of a cell or organism.

“Introduced” in the context of inserting a nucleic acid fragment (e.g., a recombinant DNA construct) into a cell, means “transfection” or “transformation” or “transduction” and includes reference to the incorporation of a nucleic acid fragment into a eukaryotic or prokaryotic cell where the nucleic acid fragment may be incorporated into the genome of the cell (e.g., chromosome, plasmid, plastid or mitochondrial DNA), converted into an autonomous replicon, or transiently expressed (e.g., transfected mRNA).

A “transformed cell” is any cell into which a nucleic acid fragment (e.g., a recombinant DNA construct) has been introduced.

“Transformation” as used herein refers to both stable transformation and transient transformation.

“Stable transformation” refers to the introduction of a nucleic acid fragment into a genome of a host organism resulting in genetically stable inheritance. Once stably transformed, the nucleic acid fragment is stably integrated in the genome of the host organism and any subsequent generation.

“Transient transformation” refers to the introduction of a nucleic acid fragment into the nucleus, or DNA-containing organelle, of a host organism resulting in gene expression without genetically stable inheritance.

The term “crossed” or “cross” means the fusion of gametes via pollination to produce progeny (e.g., cells, seeds or plants). The term encompasses both sexual crosses (the pollination of one plant by another) and selfing (self-pollination, e.g., when the pollen and ovule are from the same plant). The term “crossing” refers to the act of fusing gametes via pollination to produce progeny.

The term “introduced” means providing a nucleic acid (e.g., expression construct) or protein into a cell. Introduced includes reference to the incorporation of a nucleic acid into a eukaryotic or prokaryotic cell where the nucleic acid may be incorporated into the genome of the cell, and includes reference to the transient provision of a nucleic acid or protein to the cell. Introduced includes reference to stable or transient transformation methods, as well as sexually crossing. Thus, “introduced” in the context of inserting a nucleic acid fragment (e.g., a recombinant DNA construct/expression construct) into a cell, means “transfection” or “transformation” or “transduction” and includes reference to the incorporation of a nucleic acid fragment into a eukaryotic or prokaryotic cell where the nucleic acid fragment may be incorporated into the genome of the cell (e.g., chromosome, plasmid, plastid or mitochondrial DNA), converted into an autonomous replicon, or transiently expressed (e.g., transfected mRNA).

“Suppression DNA construct” is a recombinant DNA construct which when transformed or stably integrated into the genome of the plant, results in “silencing” of a target gene in the plant. The target gene may be endogenous or transgenic to the plant. “Silencing,” as used herein with respect to the target gene, refers generally to the suppression of levels of mRNA or protein/enzyme expressed by the target gene, and/or the level of the enzyme activity or protein functionality. The terms “suppression”, “suppressing” and “silencing”, used interchangeably herein, include lowering, reducing, declining, decreasing, inhibiting, eliminating or preventing. “Silencing” or “gene silencing” does not specify mechanism and is inclusive, and not limited to, anti-sense, cosuppression, viral-suppression, hairpin suppression, stem-loop suppression, RNAi-based approaches, and small RNA-based approaches.

Sequence alignments and percent identity calculations may be determined using a variety of comparison methods designed to detect homologous sequences including, but not limited to, the MEGALIGN® program of the LASERGENE® bioinformatics computing suite (DNASTAR® Inc., Madison, Wis.). Unless stated otherwise, multiple alignment of the sequences provided herein were performed using the Clustal V method of alignment (Higgins and Sharp, CABIOS. 5:151-153 (1989)) with the default parameters (GAP PENALTY=10, GAP LENGTH PENALTY=10). Default parameters for pairwise alignments and calculation of percent identity of protein sequences using the Clustal V method are KTUPLE=1, GAP PENALTY=3, WINDOW=5 and DIAGONALS SAVED=5. For nucleic acids these parameters are KTUPLE=2, GAP PENALTY=5, WINDOW=4 and DIAGONALS SAVED=4. After alignment of the sequences, using the Clustal V program, it is possible to obtain “percent identity” and “divergence” values by viewing the “sequence distances” table on the same program; unless stated otherwise, percent identities and divergences provided and claimed herein were calculated in this manner.

Standard recombinant DNA and molecular cloning techniques used herein are well known in the art and are described more fully in Sambrook, J., Fritsch, E. F. and Maniatis, T. Molecular Cloning: A Laboratory Manual; Cold Spring Harbor Laboratory Press: Cold Spring Harbor, 1989 (hereinafter “Sambrook”).

As will be evident to one of skill in the art, any nucleic acid of interest can be used for transformation using the methods of the present invention. Any recombinant construct comprising a nucleic acid of interest that may be introduced into a plant cell may be employed in the methods of the present invention. A plant (e.g., a soybean plant) can be engineered to express genes conferring different phenotypes, examples of such genes include, but are not limited to, disease and insect resistance genes, genes conferring nutritional value, genes to confer male and/or female sterility, antifungal, antibacterial or antiviral genes, and the like. Likewise, the method can be used to transfer any nucleic acid to control gene expression. Examples of nucleic acids that could be used to control gene expression include, but are not limited to, antisense oligonucleotides, suppression DNA constructs, or nucleic acids encoding transcription factors.

Genes of interest can be genes conferring one or more modified agronomic traits and characteristics including, but not limited to, increased yield, increased heterosis, increased oil and increased nutritional value. General categories of genes of interest include, for example, those genes involved in information, such as Zinc fingers, those involved in communication, such as kinases, and those involved in housekeeping, such as heat shock proteins. More specific categories of transgenes, for example, include genes encoding important traits for agronomics, insect resistance, disease resistance, herbicide resistance, sterility, grain characteristics and commercial products.

Agronomically important traits such as oil, starch and protein content can be genetically altered in addition to using traditional breeding methods. Modifications include increasing (or decreasing) content of oleic acid, saturated and unsaturated oils, increasing levels of lysine and sulfur and providing essential amino acids, and also modification of starch. Other proteins include methionine-rich plant proteins such as from sunflower seed (Lilley, et al. Proceedings of the World Congress on Vegetable Protein Utilization in Human Foods and Animal Feedstuffs, Applewhite, H. (ed.); American Oil Chemists Soc., Champaign, Ill.; (1989) 497-502; corn (Pedersen, et al. J. Biol. Chem. (1986) 261:6279; Kirihara et al. Gene (1988) 71:359; and rice (Musumura, et al. Plant Mol. Biol. (1989) 12:123. These references are herein incorporated by reference.

In addition to the various regulatory elements, the recombinant vector can also contain a selectable or a screenable marker, or both. The nucleic acid sequence serving as the selectable or a screenable marker functions to produce a phenotype in cells that facilitates their identification relative to cells not containing the marker. Useful selectable and screenable markers include, but are not limited to, GUS, green fluorescent protein (GFP), luciferase (LUX), antibiotic resistance sequences, and herbicide tolerance sequences.

Selectable marker genes may be utilized for the selection of transformed cells or tissues. Examples of selectable marker genes include, but are not limited to, genes encoding antibiotic resistance, such as nptII which encodes neomycin phosphotransferase II (NEO), hpt which encodes hygromycin phosphotransferase (HPT), and the moncot-optimized cyanamide hydratase gene (moCAH) (see U.S. Pat. No. 6,096,947) as well as genes conferring resistance to herbicidal compounds, such as glufosinate ammonium, bromoxynil, imidazolinones, and 2,4-dichlorophenoxyacetate (2,4-D). See generally, Yarranton (1992) Curr. Opin. Biotech. 3:506-511; Christopherson et al. (1992) Proc. Natl. Acad. Sci. USA 89:6314-6318; Yao et al. (1992) Cell 71:63-72; Reznikoff (1992) Mol. Microbiol. 6:2419-2422; Barkley et al. (1980) in The Operon, pp. 177-220; Hu et al. (1987) Cell 48:555-566; Brown et al. (1987) Cell 49:603-612; Figge et al. (1988) Cell 52:713-722; Deuschle et al. (1989) Proc. Natl. Acad. Aci. USA 86:5400-5404; Fuerst et al. (1989) Proc. Natl. Acad. Sci. USA 86:2549-2553; Deuschle et al. (1990) Science 248:480483; Gossen (1993) Ph.D. Thesis, University of Heidelberg; Reines et al. (1993) Proc. Natl. Acad. Sci. USA 90:1917-1921; Labow et al. (1990) Mol. Cell. Biol. 10:3343-3356; Zambretti et al. (1992) Proc. Natl. Acad. Sci. USA 89:3952-3956; Bairn et al. (1991) Proc. Natl. Acad. Sci. USA 88:5072-5076; Wyborski et al. (1991) Nucleic Acids Res. 19:46474653; Hillenand-Wissman (1989) Topics Mol. Struc. Biol. 10:143-162; Degenkolb et al. (1991) Antimicrob. Agents Chemother. 35:1591-1595; Kleinschnidt et al. (1988) Biochemistry 27:1094-1104; Bonin (1993) Ph.D. Thesis, University of Heidelberg; Gossen et al. (1992) Proc. Natl. Acad. Sci. USA 89:5547-5551; Oliva et al. (1992) Antimicrob. Agents Chemother. 36:913-919; Hlavka et al. (1985) Handbook of Experimental Pharmacology, Vol. 78 (Springer-Verlag, Berlin); Gill et al. (1988) Nature 334:721-724. Such disclosures are herein incorporated by reference.

The above list of selectable marker genes is not meant to be limiting. Any selectable marker gene can be used in the present invention.

Where appropriate, the gene sequence(s) may be modified to optimize for increased expression in the transformed plant. Examples of such modifications include, but are not limited to, synthesizing the genes using plant-preferred codons for improved expression. See, for example, Campbell and Gowri (1990) Plant Physiol. 92:1-11 for a discussion of host-preferred codon usage. Methods are available in the art for synthesizing plant-preferred genes. See, for example, U.S. Pat. Nos. 5,380,831, and 5,436,391 and Murray et al. (1989) Nucleic Acids Res. 17:477-498.

Additional sequence modifications are known to enhance gene expression in a cellular host. Examples of such modifications include, but are not limited to, elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

Regulatory Sequences:

A recombinant DNA construct (including a suppression DNA construct) of the present invention may comprise at least one regulatory sequence.

“Regulatory sequences” or “regulatory elements” are used interchangeably and refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include, but are not limited to, promoters, translation leader sequences, introns, and polyadenylation recognition sequences. The terms “regulatory sequence” and “regulatory element” are used interchangeably herein.

“Promoter” refers to a nucleic acid fragment capable of controlling transcription of another nucleic acid fragment.

“Promoter functional in a plant” is a promoter capable of controlling transcription in plant cells whether or not its origin is from a plant cell.

“Tissue-specific promoter” and “tissue-preferred promoter” are used interchangeably to refer to a promoter that is expressed predominantly but not necessarily exclusively in one tissue or organ, but that may also be expressed in one specific cell.

“Developmentally regulated promoter” refers to a promoter whose activity is determined by developmental events.

Promoters that cause a gene to be expressed in most cell types at most times are commonly referred to as “constitutive promoters”.

High level, constitutive expression of the candidate gene under control of the 35S or UBI promoter may have pleiotropic effects, although candidate gene efficacy may be estimated when driven by a constitutive promoter. Use of tissue-specific and/or stress-specific promoters may eliminate undesirable effects but retain the ability to enhance drought tolerance. This effect has been observed in Arabidopsis (Kasuga et al. (1999) Nature Biotechnol. 17:287-91).

Recombinant DNA constructs of the present invention may also include other regulatory sequences, including but not limited to, translation leader sequences, introns, and polyadenylation recognition sequences. In another embodiment of the present invention, a recombinant DNA construct of the present invention further comprises an enhancer or silencer.

An “intron” is an intervening sequence in a gene that is transcribed into RNA and then excised in the process of generating the mature mRNA. The term is also used for the excised RNA sequences. An “exon” is a portion of the sequence of a gene that is transcribed and is found in the mature messenger RNA derived from the gene, and is not necessarily a part of the sequence that encodes the final gene product.

“Recalcitrance” is defined herein as the inability of plant cells, tissues and organs to respond to in vitro culture. Recalcitrance can be a major limiting factor with respect to regeneration of plants (Benson, E. E. (2000) In Vitro Cell Dev Biol Plant 36:141-148). It can occur at all stages of a culture regime and little is known regarding its causal factors.

“Increased efficiency of response to tissue culture” is defined herein as decreased time to initiate response of plant cells or plant tissue to tissue culture treatments, or increased percentage generation of embryogenic tissue from somatic tissue.

“Somatic embryogenesis (SE)” is herein defined as the developmental process by which somatic cells undergo restructuring to generate embryogenic cells. These cells then go through a series of morphological and biochemical changes that result in the formation of a somatic or non-zygotic embryo capable of regenerating plants. Somatic embryogenesis represents a unique developmental pathway that includes a number of characteristic events: dedifferentiation of cells, activation of cell division, and reprogramming of their physiology, metabolism, and gene expression patterns.

Somatic embryos can differentiate either directly from the explant without an intervening callus phase or indirectly after a callus phase, referred to as direct somatic embryogenesis (DSE) and indirect somatic embryogenesis (ISE), respectively (Sharp et al., 1980 Hortic. Rev. 2: 268-310).

Embryogenic cultures capable of plant regeneration are essential for transformation by particle bombardment or Agrobacterium mediated transformation. Regeneration of mature plants via somatic embryogenesis can be divided into three stages: (1) primary induction of somatic embryos, (2) establishment of proliferative embryogenic cultures, and (3) generation of whole plants by embryo maturation and germination. Genetic variation in primary embryogenesis has been observed and studied extensively (Kita Y. et al (2007) Plant Cell Rep 26:439-447).

The lack of knowledge of the factors controlling somatic embryogenesis, the asynchrony of somatic embryo development, and low true-to-type embryonic efficiency are some of the factors that limit the application of somatic embryogenesis for regeneration of transformed plants from tissue culture.

“Regeneration,” as used herein, refers to a morphogenetic response that results in the production of new tissues, embryos, organs, whole plants or fragments of whole plants that are derived from a single cell, or a group of cells. In the present invention, the term “regeneration” encompasses production of new tissues, organs, whole plants or fragments of whole plants that are derived from a single cell, or a group of cells. Regeneration may proceed indirectly via somatic embryogenesis or directly without an intervening somatic embryo formation phase.

“Regenerative capacity” refers to the ability of a plant cell to undergo regeneration.

“Vacuum infiltration” is defined herein the process of introducing solutions into plant tissue by applying vacuum. In this procedure, plant tissue, or whole plants are submerged into the solution, followed by application and release of vacuum. Vacuum is used to extract all air bubbles inside the tissue and replace them with medium when returned to normal pressure.

“Partial Vacuum” is defined herein as a vacuum of −10 in. hg for 20 minutes or up to −28-30 in.hg per 10 minutes vacuum.

The terms “synchronization of development in tissue culture” and “synchronization of cell growth in tissue culture” are defined herein as the homogenous start of cell growth and division across the entire tissue sample treated in tissue culture.

Asynchrony of somatic embryo development and of response of plant cells, tissues or organs to tissue culture treatments is a limitation of regeneration of plants from tissue culture to be able to generate mature and fertile transgenic plants from transformed cells.

Agrobacterium-mediated gene transfer exploits the natural ability of Agrobacterium tumefaciens and A. rhizogenes to transfer DNA into plant chromosomes. Agrobacterium is a plant pathogen that transfers a set of genes encoded in a region called T-DNA of its tumor-inducing (Ti) plasmid or hairy-root-inducing (Ri) plasmid into plant cells at wound sites. This process depends on the cis acting T-DNA border sequences that flank the transferred DNA and the trans acting virulence (vir) functions encoded by the Ti plasmid or Ri plasmid and the bacteria's chromosome. The typical result of gene transfer in A. tumefaciens is a tumorous growth called a crown gall. The result of gene transfer in A. rhizogenes is hairy root disease. In both cases, gene transfer results in stable integration of the T-DNA region into a plant host chromosome. The ability to cause crown gall disease or hairy root disease can be removed by deletion of the oncogenic genes in the T-DNA without loss of DNA transfer and integration. When the oncogenic genes are removed in this manner, the Agrobacterium is said to be disarmed or non-oncogenic.

Such Agrobacterium-mediated gene transfer systems are modified to contain a heterologous or foreign nucleotide sequence of interest, such as a foreign gene or genes of interest, to be expressed in the transformed plant cells. The heterologous nucleotide sequence to be transferred is incorporated into the T-DNA region, which is flanked by imperfect 25-bp terminal repeats or T-DNA border sequences that define the end points of an integrated T-DNA. Any sequences between these terminal repeats become integrated into the plant nuclear DNA (Fraley et al. (1983) Proc. Natl. Acad. Sci. USA 80:4803; Watson et al. (1985) EMBO J. 4:277; Horsch et al. (1985) Science 227:1229; Hernalsteens et al. (1984) EMBO J. 3:3039; Comai et al. (1984) Nature 317:741; Petit et al. (1986) Mol. Gen. Genet. 202:388-393; Shah et al. (1986) Science 233:478; Bytebier et al. (1987) Proc. Natl. Acad. Sci. USA 84:5345; Schafew et al. (1987) Nature 327:529; McKnight et al. (1987) Plant Mol. Biol. 8:439-445; Potrykus (1990) Biotechnol 8:535; Grimsley et al. (1987) Nature 325:177; Gould et al. (1991) Plant Physiol. 95:426; Ishida et al. (1996) Nature Biotechnology 14:745; and U.S. Pat. No. 5,591,616, and the references cited therein).

Methods of soybean transformation based on somatic embryogenesis have been described. Embryos are induced from immature soybean cotyledons by placing the explant on high levels of 2,4-D (2,4-Dichlorophenoxyacetic acid) and the embryogenic tissues are subsequently proliferated on induction medium (Finer (1988) Plant Cell Rep 7:238-241) or liquid suspension culture (Finer and Nagasawa (1988) Plant Cell Tissue Organ Cult 15:125-136).

However, DNA delivery using particle bombardment, electroporation, or Agrobacterium-mediated delivery into soybean has proven to be difficult. This is due, in part, to the small number of cells that have been found to be totipotent in soybean (Trick et al. (1997) Plant Tissue Cult Biotechnol 3:9-26). Methods that use Agrobacterium tumefaciens for DNA delivery have the additional problem of overcoming any incompatibility between the soybean explant and the Agrobacterium.

Other methods for soybean transformation are based on particle bombardment transformation of proliferative embryogenic cultures. Fertile transgenic soybean plants have been produced using particle bombardment (Finer and McMullen (1991) In Vitro Cell Dev Biol 27P:175-182; Sato et al. (1993) Plant Cell Rep 12:408-413; Parrott et al. (1994) In Vitro Cell Dev Biol 30P:144-149; Hadi et al. (1996) Plant Cell Rep 15:500-505; Stewart et al. (1995) Plant Physiol 112:121-129; Maughan et al. (1999) In Vitro Cell Dev Biol-Plant 35:334-349). In these methods, the proliferative embryogenic cultures from both liquid and solid media are used for particle bombardment and immediate selection occurs while on solid or liquid media.

Embodiments of this invention include:

An embodiment of this invention is a method for increasing efficiency of response of plant tissue to tissue culture, the method comprising the steps of: (a) contacting a plant tissue with liquid media wherein the liquid media comprises at least one constituent selected from the group consisting of nutrients, growth regulators and antibiotics; (b) subjecting the plant tissue and liquid media of the previous step to a partial vacuum, followed by (c) returning the plant tissue and liquid media of step to atmospheric pressure; wherein there is an increased efficiency of response when compared to a plant tissue that is not subjected to a partial vacuum and further wherein the response of plant tissue can be selected from a group consisting of embryogenesis, organogenesis and cell division. In one embodiment of the invention, the liquid media contains an antibiotic and the plant tissue is not contaminated with bacteria.

In one embodiment of this invention, the efficiency of response of plant tissue to tissue culture is increased by at least 10%, by at least 20% or by at least 30% when compared to the response of a plant tissue in tissue culture that has not been subject to partial vacuum after being contacted with liquid media.

In one embodiment of this invention, the efficiency of response of plant tissue to tissue culture is increased by at least 30% when compared to the response of a plant tissue in tissue culture that has not been subject to partial vacuum after being contacted with liquid media.

Another embodiment of this invention is a method for increasing synchronization of development of plant tissue in tissue culture, the method comprising steps (a), (b) and (c) described above, wherein there is an increase in synchronization of development when compared to a plant tissue that is not subjected to a partial vacuum.

Another embodiment of this invention is a method for increasing efficiency of regeneration of a mature and fertile plant from plant tissue in tissue culture, the method comprising steps (a), (b) and (c) described above, wherein there is an increase in efficiency of regeneration when compared to a plant tissue that is not subjected to a partial vacuum.

Another embodiment of this invention is the method of increasing efficiency of response of plant tissue to tissue culture, or the method of increasing synchronization of development of plant tissue in tissue culture, or the method of increasing efficiency of regeneration of a mature and fertile plant from plant tissue in tissue culture, the method comprising the steps described above, wherein the method further comprises the step of introducing a recombinant DNA construct into the plant tissue. Furthermore, the recombinant DNA may be introduced into the plant tissue by contacting the plant tissue with Agrobacterium, or by particle bombardment.

Another embodiment of this invention is the method of increasing efficiency of response of plant tissue to tissue culture, or the method of increasing synchronization of development of plant tissue in tissue culture, or the method of increasing efficiency of regeneration of a mature and fertile plant from plant tissue in tissue culture, wherein the plant tissue is recalcitrant to tissue culture.

Another embodiment of this invention is the method of increasing efficiency of response of plant tissue to tissue culture, or the method of increasing synchronization of development of plant tissue in tissue culture, or the method of increasing efficiency of regeneration of a mature and fertile plant from plant tissue in tissue culture, wherein the plant tissue is recalcitrant to tissue culture.

In another embodiment, vacuum infiltration of plant tissues may be done to introduce different solutions to synchronize cell growth.

In another embodiment, vacuum infiltration can be used to introduce chelated molecules for a slow but continue release of active agents across the tissues.

In another embodiment, vacuum infiltration during plant tissue culture can be used to introduce protein bound active molecules to target specific organs.

In another embodiment, vacuum infiltration can be used to introduce nanoparticles embedded with different molecules into plant tissues.

In another embodiment, vacuum infiltration can be used to create more uniform tissue dehydration for a better response with particle gun bombardment.

In another embodiment, vacuum infiltration can be used introduce tissue staining agents.

In another embodiment, the methods disclosed in the current invention are used for increasing the tissue culture response of soybean plant tissues.

In another embodiment, the invention includes plant cells, tissues, plants, and seeds generated by using the methods disclosed in the current invention. The invention encompasses regenerated, mature and fertile plants, seeds produced therefrom, T1 and subsequent generations. The plant cells, tissues, plants and seeds may be soybean cells, tissues, plants and seeds.

### EXAMPLES

The present invention is further illustrated in the following Examples, in which parts and percentages are by weight and degrees are Celsius, unless otherwise stated. It should be understood that these examples, while indicating embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Furthermore, various modifications of the invention in addition to those shown and described herein will be apparent to those skilled in the art from the foregoing description. Such modifications are also intended to fall within the scope of the appended claims.

### Example 1

### Method for Synchronizing Cell Growth and Increasing Somatic Embryogenesis Efficiency in Soybean Tissue Culture

A method was developed for synchronizing cell growth in soybean tissue culture producing a suitable tissue for transformation faster. The method comprises the steps of homogenously introducing cell growth medium inside the plant samples from immature soybean seeds, inducing primary and secondary somatic embryogenesis, followed by regenerating plantlets from the transformed and not transformed tissue. Immature soybean seeds were surface sterilized according the standard seed sterilization protocol. Following removal of the seed coat, immature cotyledons were placed into liquid cell growth (induction) medium without antibiotics. Cotyledons were vacuum infiltrated up to 28 in. Hg for up to 20 minutes. Following vacuum infiltration, the liquid induction medium was removed and cotyledons were plated onto solid induction medium without antibiotics for proliferation of primary and secondary somatic embryos.

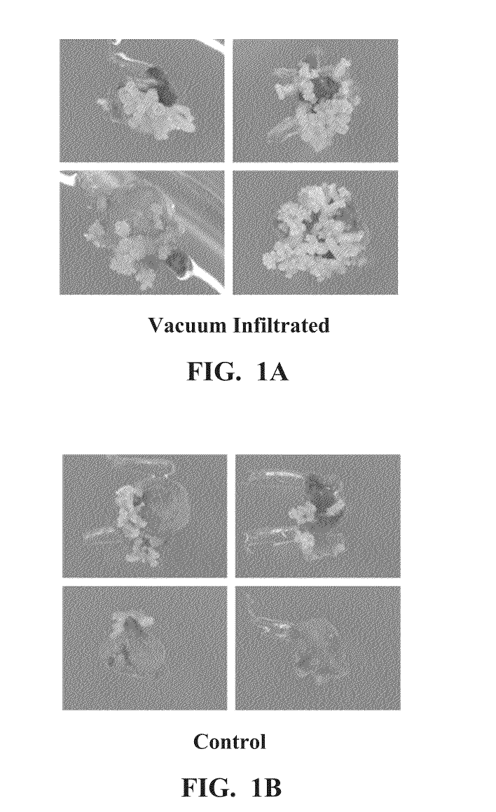

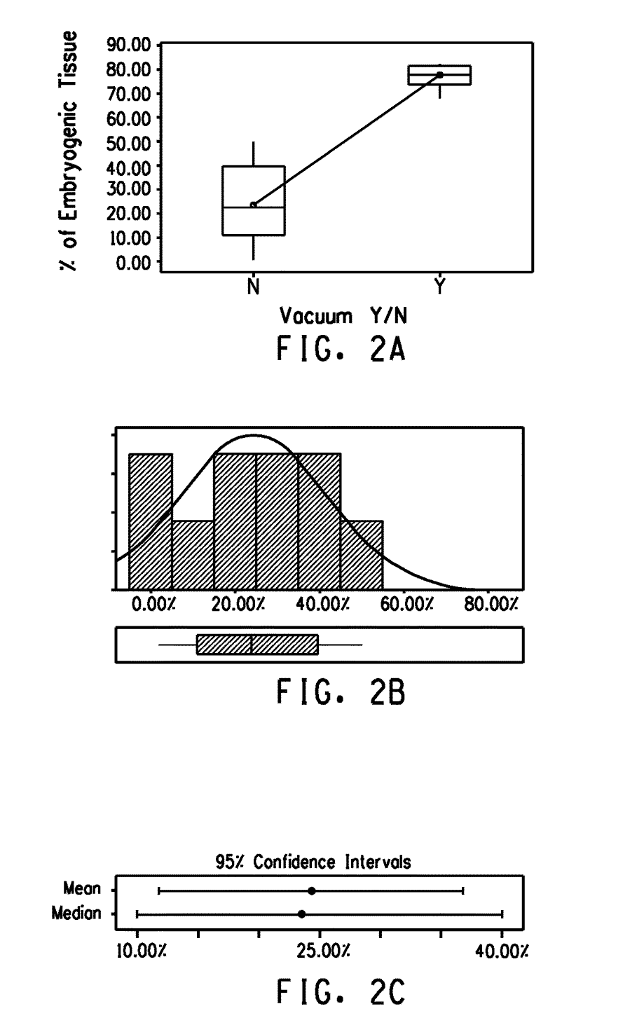

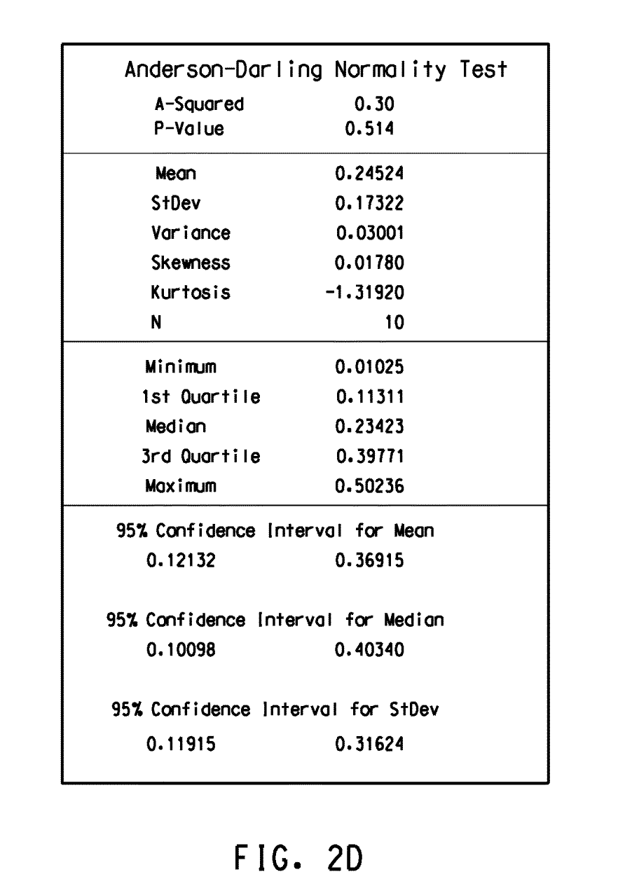

FIG. 1A-1B and FIG. 2A-2D show the increase in somatic embryogenesis after vacuum infiltration.

### Example 2

### Use of Vacuum Infiltration Method for Controlling Contamination in Plant Samples

The vacuum infiltration method can be used for preventing and/or controlling endogenous bacteria growing inside the plant samples that are not reachable with the established sterilization procedure. Immature soybean seeds were surface sterilized following the standard seed sterilization protocol. Before removal of the seed coat, immature seeds were surface sterilized with ethanol solution with and without the use of surfactant. Following the ethanol rinse, the seed coat was removed and immature cotyledons were harvested. No additional rinses were used to remove any residual ethanol solution. Cotyledons were either plated directly onto solid induction medium (Samoylov, V. M. et al (1998) In Vitro Cell Dev. Biol.-Plant 34:8-13.) or floated in liquid induction medium and vacuum infiltrated as previously described in Example 1. Soybean tissue from Cv Jack and 93886 was also vacuum infiltrated with liquid induction medium containing antibiotics. Following removal of the seed coat immature cotyledons were placed into liquid cell growth (induction) medium. The liquid induction medium contained cefotaxime, carbenicillin, or a combination of both antibiotics at various concentrations (Table 1). The vacuum infiltration was done at a pressure of −28 in. Hg with various medium (PDA, TSA and SB103) and with the same media with combinations of Carbenicillin and Cefotaxime (Sigma) according to the DOE shown in Table 1. The vacuum was applied using a Vacuum Desiccator Cabinet (Labconco) at −28 in. Hg.

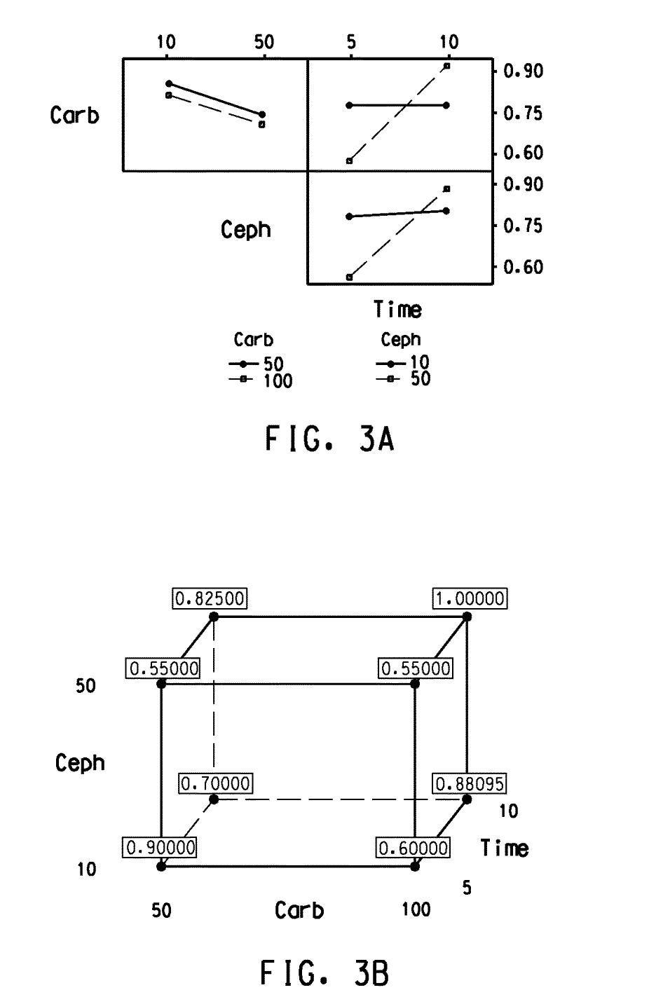

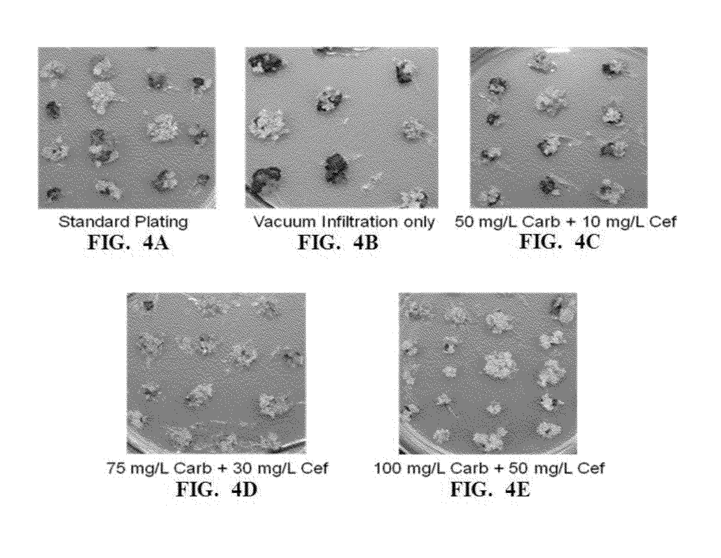

Following vacuum infiltration, the liquid induction medium was removed and cotyledons were plated onto solid induction medium for proliferation of primary and secondary somatic embryos. Following somatic embryogenesis, individual cotyledons were macerated and the cellular contents plated onto solid bacterial screening medium (FIG. 3A-3B and FIG. 4A-4E). Following a two-to-three-week incubation period in darkness at room temperature, no sign of bacterial growth was detected. An alternative screening method involves macerating individual cotyledons in liquid bacterial screening medium and incubating them at an elevated temperature while shaking.

TABLE 1
A Placket-Burman Design on 3 factors (Carbenicillin, Cephotaxime and Time)

| Std | Run | Pt | | Carbenicillin | Cephotaxime | Time | % |
|---|---|---|---|---|---|---|---|
| Order | Order | Type | Blocks | (mg/L) | (mg/L) | (mins) | Response |
| 4 | 1 | 1 | 1 | 100 | 10 | 10 | 76.19% |
| 5 | 2 | 1 | 1 | 100 | 50 | 5 | 55.00% |
| 1 | 3 | 1 | 1 | 100 | 10 | 10 | 100.00% |
| 11 | 4 | 1 | 1 | 50 | 50 | 5 | 55.00% |
| 10 | 5 | 1 | 1 | 100 | 10 | 5 | 60.00% |
| 7 | 6 | 1 | 1 | 50 | 50 | 10 | 75.00% |
| 6 | 7 | 1 | 1 | 100 | 50 | 10 | 100.00% |
| 12 | 8 | 1 | 1 | 50 | 10 | 5 | 85.00% |
| 9 | 9 | 1 | 1 | 50 | 10 | 5 | 95.00% |
| 8 | 10 | 1 | 1 | 50 | 10 | 10 | 70.00% |
| 2 | 11 | 1 | 1 | 100 | 50 | 5 | 55.00% |
| 3 | 12 | 1 | 1 | 50 | 50 | 10 | 90.00% |

2,4-D might be used as the growth regulator in this protocol. Treated samples were dried on filter paper and then transferred onto plates of solid callus induction SB199-medium given below. After 2 weeks the samples were transferred into bacterial screening medium in a 14-ml round bottom culture tubes to detect bacterial presence.

### Composition of SB199 Media:

### Dissolve Ingredients in Polished d.i. h20 in Sequence

| Ingredient | Quantity | |
|---|---|---|
| Nanopure water | 950.00000 | ml |
| MS basal salt mixture | 4.33000 | g |
| B5 vitamins 1000X stock (13135 BASE) | 1.00000 | ml |
| 2,4-D (10 mg/ml) | 4.00000 | ml |
| Sucrose | 30.00000 | g |
| Q.S. to volume. Adjust pH to 7.0. | | |
| Post Q.S. additives | | |
| Gelrite gellan gum | 2.00000 | g |

Take pH measurement, sterilize in Agarclay. Pour designated volume per container.

Treatment response was recorded and analyzed for the most effective treatment(s) with Minitab 15 (Minitab inc.): results are shown in Table 1 and FIG. 3 and FIG. 4. The digital pictures of the treated tissues were analyzed with AxioVision software on Axiophot microscopes (Zeiss). The percentage response refers to the percentage of samples that were not contaminated.

### Protocol for Contamination Control Using Vacuum Infiltration:

Bleach sterilized seeds were put into an empty Petri plate (100×25 mm). Seeds were soaked in 70% ethanol for 30 seconds. Ethanol was drawn off with a pipette. Embryo isolation was continued in the same plate.

After making a 4 mm mark on the center plate of the microscope, the seeds were measured. Only those that were less than 4 mm in length were used. The rounded end of the seed was cut and the cotyledons were pressed out of the seed coat. The isolated cotyledons were gathered on an SB199 plate to avoid over-drying.

The required amount of SB199 liquid medium was prepared with the final concentration of Carbenicillin 100 mg/L and Cefotaxime 50 mg/L. Typical media volume used was 10 ml.

Liquid medium was added to a Petri plate (60×15 mm) and isolated cotyledons were added to the medium and lid was replaced. Small Petri plate was placed into a large 100×25 mm Petri plate. In a vacuum chamber (either free standing or gene gun chamber) vacuum (22-28 psi) was pulled on the cotyledons for 10 minutes.

Liquid medium was removed from the plate. Cotyledons were placed on SB199 solid medium with the inner flat side up, around 25 cotyledons on each plate. The plates were wrapped with fiber tape and were placed on dedicated initiation shelves (˜60 microEinstein/m²/s). Two weeks after initiation, the explants were transferred to SB1 medium, same orientation.

In 3-4 weeks, secondary embryos were cut and put into SB196 liquid medium. One plate of embryos was cut into one flask (best practice). In 7-10 days, the proliferative secondary embryos were subcultured and selected. The composition of the SB196 media is given below:

| Ingredient | Quantity | |
|---|---|---|
| Nanopure water | 950.00000 | ml |
| Potassium phosphate monobasic anhydrous | 18.50000 | g |
| Boric Acid | 0.62000 | g |
| 2,4-D (10 mg/ml) | 4.00000 | ml |
| Sodium Molybdate•2H₂O | 0.02500 | g |
| Q.S. to volume. | | |

Filter Sterilize through a 0.22μ filter into a 1 L filter unit.

Bacterial Indexing Step was performed (in triplicate per flask): 3 clumps of embryos per flask were selected. Each tissue sample was macerated or crushed in 2.5 ml of liquid 523 Bacterial Screening Medium in a separate 14 ml round-bottom culture tube. Tubes were incubated in the dark at 30° C. on a shaker at 250 rpm. Culture tubes were observed in 3 to 5 days for bacterial growth.

To the remaining stock culture material, SB196 was added with the final concentration of Carbenicillin 100 mg/L and Cefotaxime 50 mg/L.

In a vacuum chamber (either free standing or gene gun chamber) pulled vacuum (22-28 psi) on the culture for 10 minutes. Antibiotic-spiked media was decanted and culture was refreshed with ˜60 ml SB196 medium. Held cultures until results from the bacterial indexing were available (3-5 days). If the growth was not optimal, subculturing was done for an additional week before distribution.

We claim:

1. A method for increasing efficiency of response of plant tissue to tissue culture, the method comprising the steps of:
a) contacting a plant tissue with liquid media wherein the liquid media comprises at least one constituent selected from the group consisting of nutrients, growth regulators and antibiotics,
b) subjecting the plant tissue and liquid media of step (a) to a partial vacuum, and
c) returning the plant tissue and liquid media of step (b) to atmospheric pressure,
wherein there is an increased efficiency of response when compared to a plant tissue that is not subjected to a partial vacuum and further wherein the response of plant tissue can be selected from a group consisting of embryogenesis, organogenesis and cell division.

2. A method for increasing synchronization of development of plant tissue in tissue culture, the method comprising the steps of:
a) contacting a plant tissue with liquid media wherein the liquid media comprises at least one constituent selected from the group consisting of nutrients, growth regulators and antibiotics,
b) subjecting the plant tissue and liquid media of step (a) to a partial vacuum, and
c) returning the plant tissue and liquid media of step (b) to atmospheric pressure;
wherein there is an increase in synchronization of development when compared to a plant tissue that is not subjected to a partial vacuum.

3. A method for increasing efficiency of regeneration of a mature and fertile plant from plant tissue in tissue culture, the method comprising the steps of:
a) contacting a plant tissue with liquid media wherein the liquid media comprises at least one constituent selected from the group consisting of nutrients, growth regulators and antibiotics,
b) subjecting the plant tissue and liquid media of step (a) to a partial vacuum, and
c) returning the plant tissue and liquid media of step (b) to atmospheric pressure;
wherein there is an increase in efficiency of regeneration when compared to a plant tissue that is not subjected to a partial vacuum.

4. The method of claim 1, 2 or 3, further comprising the step of introducing a recombinant DNA construct into the plant tissue.

5. The method of claim 4 wherein the recombinant DNA construct is introduced into the plant tissue by contacting the plant tissue with Agrobacterium.

6. The method of claim 4 wherein the recombinant DNA construct is introduced into the plant tissue by particle bombardment.

7. A stable transformed plant obtained by the method of claim 4, 5 or 6, wherein the stable transformed plant comprises all or part of the recombinant DNA construct.

8. A progeny plant or seed obtained from the plant of claim 7, wherein the progeny plant or seed comprises all or part of the recombinant DNA construct.

9. The method of claim 1 wherein the efficiency of response of plant tissue to tissue culture is increased by at least 30% when compared to the response of a plant tissue in tissue culture that has not been subject to partial vacuum after being contacted with liquid media.

10. The method of claim 1, 2 or 3 wherein the plant tissue is recalcitrant to tissue culture.

11. The method of claim 1, 2 or 3 wherein the liquid media contains an antibiotic and the plant tissue is not contaminated with bacteria.

12. A plant obtained by the method of claim 1, 2, 3, 9, 10 or 11.

13. A progeny plant or seed obtained from the plant of claim 12.
